(19) United States
(12) Patent Application Publication
(10) Pub. No.: US 2026/0327398 A1
(43) Pub. Date: Oct. 22, 2026

(54) IONIC LIQUID-CATALYZED GLYCOLYSIS OF POLYETHYLENE TEREPHTHALATE

(71) Applicant: King Fahd University of Petroleum and Minerals, Dhahran (SA)

(72) Inventors: Mamdouh A. AL-HARTHI, Dhahran (SA); Hassam MAZHAR, Hangzhou (CN); Akmuhammet Karayev, Dhahran (SA)

(73) Assignee: King Fahd University of Petroleum and Minerals, Dhahran (SA)

(21) Appl. No.: 19/266,833

(22) Filed: Jul. 11, 2025

Related U.S. Application Data
(60) Provisional application No. 63/790,523, filed on Apr. 17, 2025.

(51) Int. Cl. C08J 11/10; C07C 51/347; C07C 63/26; C08G 63/183; C08K 5/053; C08K 5/3445; C08K 5/50

(52) U.S. Cl. CPC C08J 11/10; C07C 51/347; C07C 63/26; C08G 63/183; C08K 5/053; C08K 5/3445; C08K 5/50; C08J 2367/03

(57) ABSTRACT

A method of polyethylene terephthalate glycolysis includes refluxing a refluxing a glycolysis mixture comprising a polyethylene terephthalate (PET) with ethylene glycol and an ionic liquid at a temperature of 100 to 300° C. to form a reaction mixture. The polyethylene terephthalate is from post-consumer water bottles, the ionic liquid is 1-butyl-3-methylimidazolium acetate ([BMIM][OAc]) and/or trihexyltetradecylphosphonium decanoate ([P6,6,6,14][Dec]). The ionic liquid is present in the reaction mixture in an amount of 2 to 15 percent by weight (wt. %) of the PET. The method further includes filtering the reaction mixture to collect a filtrate and cooling the filtrate to form bis(2-hydroxyethyl) terephthalate (BHET) where at least 95 percent by weight (wt. %) of the PET is converted to BHET.

### CROSS REFERENCE TO RELATED APPLICATION

This application claims the benefit of U.S. Provisional Application No. 63/790,523, filed Apr. 17, 2025, which is incorporated herein by reference in its entirety.

### BACKGROUND

### Technical Field

The present disclosure relates to polymer depolymerization and chemical recycling. More specifically, the present disclosure pertains to a method of glycolytic depolymerization of polyethylene terephthalate (PET) using ionic liquids as metal-free catalysts to recover bis(2-hydroxyethyl) terephthalate (BHET) from post-consumer PET sources.

### Description of Related Art

The “background” description provided herein is for the purpose of generally presenting the context of the disclosure. The work of the presently named inventors, to the extent it is described in this background section, as well as aspects of the description which may not otherwise qualify as prior art at the time of filing, are neither expressly nor impliedly admitted as prior art against the present disclosure.

The widespread use of plastics has resulted in environmental and health challenges due to insufficient plastic waste management. Global plastic production is estimated to reach 311 million tons annually [Bornscheuer, U. T., Feeding on plastic, Science, 2016, 351, 1154-1155]. Between 1950 and 2015, approximately 12% of the plastic waste was incinerated, while only 9% underwent recycling processes. This suggests that most plastic is likely discarded through other means, such as landfill disposal or accumulation in the environment [Geyer, R., Chapter 2-Production, use, and fate of synthetic polymers, in: T.M.B.T.-P.W. and R. Letcher (Ed.), Academic Press, 2020, pp. 13-32]. The inert and largely non-biodegradable nature of plastics hinder their decomposition and reintegration into the natural cycle with the process of decomposition spanning several centuries [Shojaei, B. et al., Chemical recycling of PET: A stepping-stone toward sustainability, Polym. Adv. Technol., 2020, 31, 2912-2938].

Waste polyethylene terephthalate (PET), which accounts for 10% of the total plastic waste, has become an environmental concern due to its extensive applications in the packaging industry and its resistance to natural degradation. In 2019, the global production capacity of PET was approximately 30.5 million metric tons. By 2024, this quantity was projected to reach nearly 35.3 million metric tons [Jaganmohan, M., Global production capacity of polyethylene terephthalate 2014-2024, Statistica, 2021]. Accumulation of waste PET in landfills and its disposal into oceans pose serious ecological issues, demanding innovative strategies to facilitate sustainable solutions.

PET is a condensation polymer that is generated by the polymerization of terephthalic acid (TPA) and ethylene glycol (EG) (FIG. 2) or through transesterification of dimethyl terephthalate (DMT) and ethylene glycol (FIG. 3). DuPont developed PET on a commercial scale in the mid-1940s, and it quickly became a widely produced thermoplastic material [Kint, D. and Muñoz-Guerra, S., A review on the potential biodegradability of poly(ethylene terephthalate), Polym. Int., 1999, 48, 346-352Webb, H. K. et al., Plastic degradation and its environmental implications with special reference to poly(ethylene terephthalate), Polymers (Basel), 2012, 5, 1-18; and Awaja, F. and Pavel, D., Recycling of PET, Eur. Polym. J., 2005, 41, 1453-1477]. PET is used in the textile industry in the form of fibers, which are subsequently utilized in the manufacturing of polymer films. In addition, the polymer has been widely employed in the injection blow molding processes for the purpose of manufacturing robust and transparent bottles and jars [Rosato, D. V., Plastics processing data handbook, Springer Science & Business Media, 2012; and Karayannidis, G. P. and Psalida, E. A., Chain extension of recycled poly(ethylene terephthalate) with 2, 2′-(1, 4-phenylene) bis(2-oxazoline), J. Appl. Polym. Sci., 2000, 77, 2206-2211]. PET plastic bottles, which were patented by engineer Nathaniel Wyeth in 1973, saw rapid market adoption, have garnered popularity and adaptability on a global scale, and have risen as a popular option for beverage containers.

Growth in the PET market is driven by its advantages of high durability, transparency, lightweight design, flexibility, non-reactivity, affordability, strong pressure resistance, mechanical strength, and effective barrier properties that prevent liquid and gas permeability [Hiraga, K. et al., Biodegradation of waste PET: A sustainable solution for dealing with plastic pollution, EMBO Rep., 2019, 20, e49365].

Introduction of millions of metric tons of plastic waste into the ocean has resulted in the formation of an extensive plastic accumulation, referred to as “the Great Pacific garbage patch.” This influx of plastic debris presents a threat to the lives of marine animals [Lamb, J. B. et al., Plastic waste associated with disease on coral reefs, Science, 2018, 359, 460-462; and Li, W. C. et al., Plastic waste in the marine environment: A review of sources, occurrence and effects, Sci. Total Environ., 2016, 566, 333-349]. Plastics have been shown to pollute the soil environment, causing detrimental effects on soil composition, water potential, nutrient levels, and terrestrial biodiversity [Wang, J. et al., Microplastics as contaminants in the soil environment: A mini-review, Sci. Total Environ., 2019, 691, 848-857]. Internalization of nanoparticles of PET by macrophage cell lines leads to oxidative stress and disruptions in cell multiplication and growth, demonstrating the health hazards linked to inhaling and ingestion of PET [Aguilar-Guzmán, J. C. et al., Polyethylene terephthalate nanoparticles effect on RAW 264.7 macrophage cells, Microplastics and Nanoplastics, 2022, 2, 1-15].

As PET waste continues to grow, a circular economy offers a practical solution to address energy and resource use, reduce harmful emissions, and decrease the amount of poorly managed plastic waste entering the environment [Prata, J. C. Et al., Solutions and integrated strategies for the control and mitigation of plastic and microplastic pollution, Int. J. Environ. Res. Public Health, 2019, 16, 2411].

There are several PET recycling methods with advantages and limitations. Mechanical recycling is one of the most cost-effective methods and is widely used in industry. This process involves collecting, washing, separating, and re-melting of PET to form pellets. This recycling method is considered a temporary solution because the quality of the recycled PET (rPET) drops with each cycle and is often used for secondary materials that eventually end up in landfills [Frounchi, M. et al., Studies on degradation of PET in mechanical recycling, Macromol. Symp., 1999, 144, 465-469].

Chemical recycling methods (hydrolysis, methanolysis, aminolysis, and glycolysis) provide raw materials that can be re-polymerized into high-quality PET. Hydrolysis breaks PET down into ethylene glycol (EG) and terephthalic acid (TPA) but is less commonly used for food-grade recycling due to the high purification costs of TPA and it requires extreme conditions of 1.4-2 MPa pressure and temperatures of 200-250° C. in either acidic, basic, or neutral environments [Everette, M. R., Recovery of methyl esters of aromatic acids and glycols from thermoplastic polyester scrap, 1991]. Methanolysis involves heating PET in methanol at 180-280° C. under high pressure (2-4 MPa) to maintain methanol in liquid form. Though effective, it produces a mixture of by-products and relies on toxic reagents, making purification costly, with a typical yield of 80-85% dimethylterephthalate (DMT) [Paul, B. K. et al., Process for converting pet scrap to diamine monomers, 1989]. Aminolysis, a lesser-studied method, involves reacting PET with anhydrous amines in EG to form terephthalamide, which can be further processed into other compounds. This method operates at 120-180° C. and 2 MPa but often creates complex by-products that complicate purification [Paszun, D. and Spychaj, T., Chemical recycling of poly(ethylene terephthalate), Ind. Eng. Chem. Res., 1997, 36, 1373-1383; Spychaj, T., Chemical recycling of PET: methods and products, Handb. Thermoplast. Polyesters Homopolymers, Copolym. Blends, Compos., 2002, 1252-1290; and D. Cornell, Depolymerization of PET for food packaging, Proc. Conf. Recycl. RETEC, Soc. Plast. Eng., 1993].

Among chemical recycling processes, glycolysis is a pragmatic and effective procedure. It operates under milder conditions, making it more energy-efficient [Mendiburu-Valor, E. et al., Valorization of urban and marine PET waste by optimized chemical recycling, Resour. Conserv. Recycl., 2022, 184, 106413]. Glycolysis uses ethylene glycol (EG) as a stable depolymerizing agent and a solvent at the same time, resulting in fewer by-products. This process yields bis(2-hydroxyethyl) terephthalate (BHET), a valuable monomer reusable in PET synthesis and versatile for producing textile dyes, polyurethane oligomers, foams, biocompatible materials, and other related applications [Westover, C. C. and Long, T. E., Envisioning a BHET Economy: Adding Value to PET Waste, Sustain. Chem., 2023, 4, 363-393].

PET glycolysis is catalyzed by compounds like metal salts, ionic liquids, and enzymes. The reaction mechanism typically involves a hydroxyl group of ethylene glycol attacking a carbonyl carbon of an ester in the PET to form bis(2-hydroxyethyl) terephthalate (BHET) and release ethylene glycol.

Metal salts like zinc acetate offer effective catalytic activity but often require long reaction times and high energy input. Enzyme-based glycolysis is environmentally friendly but hasn't been implemented in large-scale operations due to the unpredictable nature of the process [Xin, J. et al., Progress in the catalytic glycolysis of polyethylene terephthalate, J. Environ. Manage, 2021, 296, Other catalyst-free approaches like microwave-assisted glycolysis increase reaction efficiency, but implementation in industry depends on development of a large-scale continuous microwave reactor [Barnard, E. et al., Chemolytic depolymerisation of PET: a review, Green Chem., 2021, 23, 3765-3789].

Despite widespread use of metallic catalysts in the PET glycolysis and their catalytic performance, they are hindered by high costs, inadequate durability, and negative environmental impacts [Liu, X. and Dai, L., Carbon-based metal-free catalysts, Nat. Rev. Mater., 2016, 1, 1-12; and Rasool, B. et al., Impacts of oxalic acid-activated phosphate rock and root-induced changes on Pb bioavailability in the rhizosphere and its distribution in mung bean plant, Environ. Pollut., 2021, 280, 116903].

An object of the present disclosure is to provide a method of polyethylene terephthalate glycolysis that may circumvent drawbacks and limitations of materials and methods known in the art.

### SUMMARY

In an exemplary embodiment, a method of polyethylene terephthalate glycolysis is described. The method includes refluxing a refluxing a glycolysis mixture comprising a polyethylene terephthalate (PET) with ethylene glycol and an ionic liquid at a temperature of 100 to 300° C. to form a reaction mixture. The polyethylene terephthalate is from post-consumer water bottles, the ionic liquid is selected from a group consisting of 1-butyl-3-methylimidazolium acetate ([BMIM][OAc]) and trihexyltetradecylphosphonium decanoate ([P6,6,6,14][Dec]), and the ionic liquid is present in the reaction mixture in an amount of 2 to 15 percent by weight (wt. %) of the PET. The method further includes filtering the reaction mixture to collect a filtrate and cooling the filtrate to form bis(2-hydroxyethyl) terephthalate (BHET). At least 95 percent by weight (wt. %) of the PET is converted to BHET.

In some embodiments, the ionic liquid is [BMIM][OAc] and the BHET has a yield percentage of 30 to 35% based on an initial weight of the PET.

In some embodiments, the ionic liquid is [P6,6,6,14][Dec] and the BHET has a yield percentage of 70 to 75% based on an initial weight of the BHET.

In some embodiments, the refluxing occurs for 1 to 5 hours.

In some embodiments, the cooling occurs at a temperature of 2 to 6° C.

In some embodiments, the cooling occurs for 16 to 24 hours.

In some embodiments, the ionic liquid is present in the reaction mixture in an amount of 4 to 6 wt. % of the PET.

In some embodiments, the method further includes mixing the BHET to a temperature of 150 to 250° C. under nitrogen to melt the BHET and heating the melted BHET to a temperature of 250 to 300° C. under vacuum for 40 to 60 minutes to form a polycondensation product. The polycondensation product is the polyethylene terephthalate (PET).

In some embodiments, the ionic liquid is trihexyltetradecylphosphonium bis(2,4,4-trimethylpentyl)phosphinate ([P6,6,6,14][TMPP]) and 55 to 65% of the PET is converted to the BHET based on an initial weight of the PET.

In some embodiments, the refluxing occurs at 0.8 to 1.2 bar.

In some embodiments, a weight ratio of the PET to the ethylene glycol during the refluxing is 1-3:20-25.

In some embodiments, at least 97 wt. % of the PET is converted to BHET.

In some embodiments, at least 99 wt. % of the PET is converted to BHET.

In some embodiments, the ionic liquid is [BMIM][OAc] and the BHET has a yield percentage of 33 to 34% based on an initial weight of the PET.

In some embodiments, the ionic liquid is [P6,6,6,14][Dec] and the BHET has a yield percentage of 72 to 73% based on an initial weight of the BHET.

In some embodiments, the reaction mixture during the refluxing is metal-free.

In some embodiments, the ionic liquid is trihexyltetradecylphosphonium bis(2,4,4-trimethylpentyl)phosphinate ([P6,6,6,14][TMPP]) and 59 to 61% of the PET is converted to the BHET based on an initial weight of the PET.

In some embodiments, the refluxing occurs at a temperature of 150 to 250° C.

In some embodiments, the BHET is repolymerized into the PET.

In some embodiments, the refluxing occurs at a temperature of 190 to 210° C.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a flow chart depicting a method of polyethylene terephthalate glycolysis, according to certain embodiments.

FIG. 1B is a schematic diagram of waste polyethylene terephthalate (PET) handling, according to certain embodiments.

FIG. 2 illustrates a reaction pathway for formation of PET from ethylene glycol (EG) and terephthalic acid (TPA), according to certain embodiments.

FIG. 3 illustrates a transesterification reaction between dimethyl terephthalate (DMT) and

EG to form PET, according to certain embodiments.

FIG. 4 depicts a reaction equation for the glycolysis of PET into bis(2-hydroxyethyl) terephthalate (BHET), according to certain embodiments.

FIG. 5 is a schematic illustration of a PET glycolysis reaction setup, according to certain embodiments.

FIG. 6A depicts the molecular structure of 1-butyl-3-methylimidazolium acetate ([BMIM][OAc]), according to certain embodiments.

FIG. 6B depicts the molecular structure of trihexyltetradecylphosphonium bis(2,4,4-trimethylpentyl)phosphinate ([P6,6,6,14][TMPP]), according to certain embodiments.

FIG. 7 is a proton nuclear magnetic resonance (¹H NMR) spectrum of [BMIM][OAc], according to certain embodiments.

FIG. 8 is a carbon-13 nuclear magnetic resonance (13C NMR) spectrum of [BMIM][OAc], according to certain embodiments.

FIG. 9 illustrates catalytic effects of various ionic liquids on the glycolysis of PET at 200° C. with 5 wt. % catalyst concentration and 3-hour reaction time, according to certain embodiments.

FIG. 10 is a Fourier-transform infrared (FTIR) spectrum of a glycolysis product, according to certain embodiments.

FIG. 11A is an 1H NMR spectrum of the glycolysis product, according to certain embodiments.

FIG. 11B is a 13C NMR spectrum of the glycolysis product, according to certain embodiments.

FIG. 12A is a gas chromatography (GC) spectrum from a gas chromatography-mass spectrometry (GC-MS) analysis of the glycolysis product, according to certain embodiments.

FIG. 12B is a mass spectrum from the GC-MS analysis, depicting fragment ions corresponding to BHET, according to certain embodiments.

FIG. 13A illustrates an FTIR analysis of BHET, original PET, and a re-polymerized product, according to certain embodiments.

FIG. 13B illustrates thermogravimetric analysis (TGA) results of PET, BHET, and the re-polymerized product, according to certain embodiments.

FIG. 14A depicts a solid-state 13C NMR spectrum of the polycondensation product, according to certain embodiments.

FIG. 14B is a solid-state 13C NMR spectrum of the original PET, according to certain embodiments.

### DETAILED DESCRIPTION

When describing the present disclosure, the terms used are to be construed in accordance with the following definitions, unless a context dictates otherwise.

Embodiments of the present disclosure will now be described more fully hereinafter with reference to the accompanying drawings wherever applicable, in that some, but not all, embodiments of the disclosure are shown.

In the drawings, like reference numerals will be used to designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “room temperature” refers to a temperature range of 25±3 degrees Celsius (° C.).

As used herein, the term “polyethylene terephthalate glycolysis” refers to a chemical recycling process in which polyethylene terephthalate (PET) is broken down into its monomeric units using a glycol, typically under heat and in the presence of a catalyst.

As used herein, the term “depolymerization process” refers to a chemical breakdown of a polymer into monomers or oligomers through the cleavage of chemical bonds.

As used herein, the term “refluxing” refers to a process of heating a reaction mixture while continuously condensing and returning a vapor back to the reaction flask to maintain a constant reaction temperature.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers.

Aspects of the present disclosure are directed to ionic liquid-assisted glycolysis of polyethylene terephthalate (PET) to bis(2-hydroxyethyl) terephthalate (BHET) with high conversion and selectivity, followed by successful re-polymerization back to PET.

A method of polyethylene terephthalate glycolysis is described. Polyethylene terephthalate (PET) glycolysis is a depolymerization process in which PET plastic is chemically broken down into its monomeric units using a glycol. During this reaction, ester linkages in the PET polymer chains are cleaved through transesterification, resulting in the formation of bis(2-hydroxyethyl) terephthalate (BHET). The process is usually carried out at elevated temperatures under reflux conditions and can be enhanced by the use of catalysts, such as ionic liquids or metal salts. Glycolysis is an efficient and sustainable method for recycling PET, as the resulting monomers can be purified and reused to synthesize new PET, enabling closed-loop recycling.

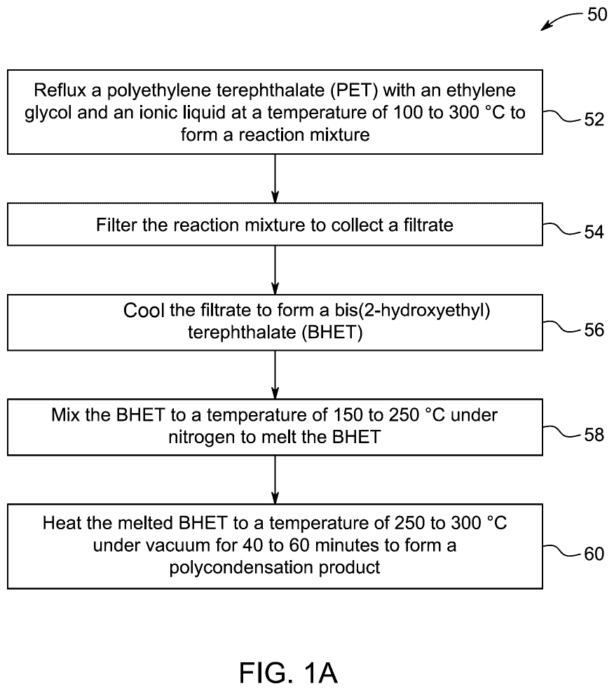

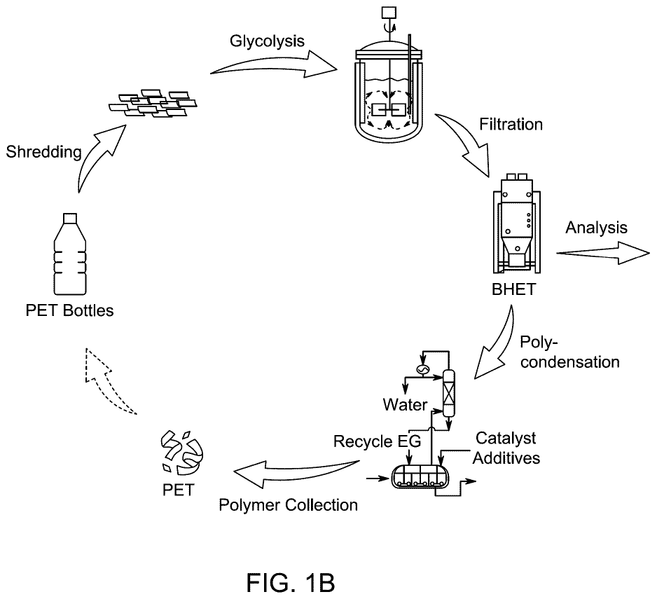

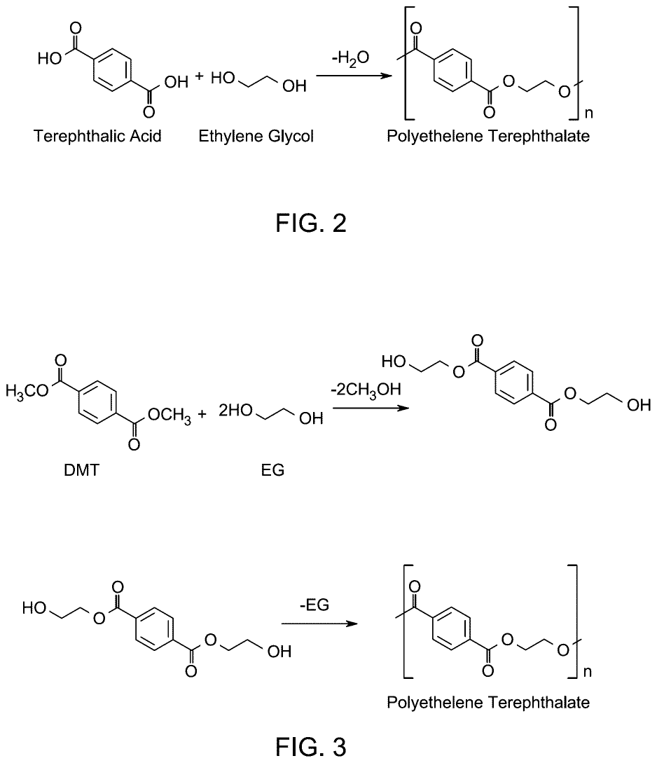

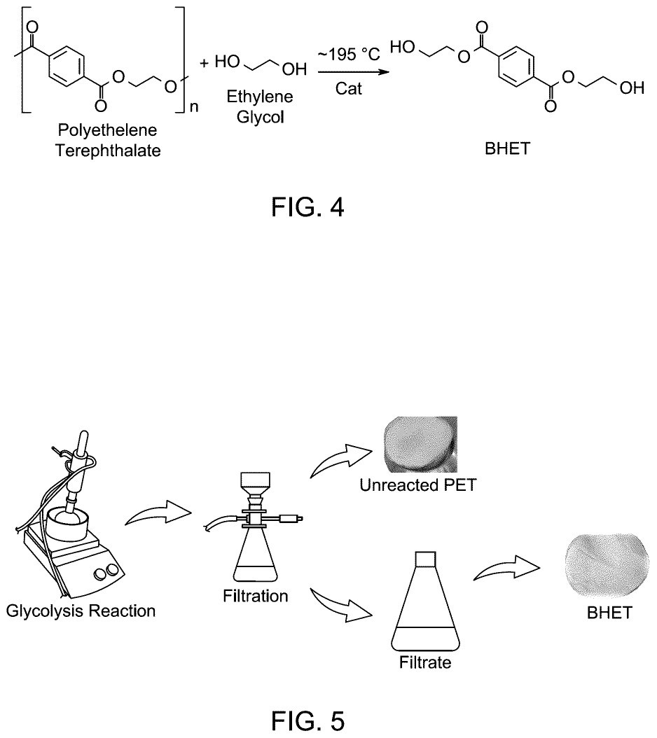

A method of polyethylene terephthalate glycolysis is described. FIG. 1A illustrates a schematic flow chart of a method 50 of polyethylene terephthalate glycolysis. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes refluxing a refluxing a glycolysis mixture comprising a polyethylene terephthalate (PET) with ethylene glycol and an ionic liquid at a temperature of 100 to 300° C., preferably 110 to 290° C., preferably 120 to 280° C., preferably 130 to 270° C., preferably 140 to 260° C., preferably 150 to 250° C., preferably 160 to 240° C., preferably 170 to 230° C., preferably 180 to 220° C., more preferably 190 to 210° C., and yet more preferably about 200° C. to form a reaction mixture. In some embodiments, the refluxing occurs at a temperature of 150 to 250° C., preferably 155 to 245° C., preferably 160 to 240° C., preferably 165 to 235° C., preferably 170 to 230° C., preferably 175 to 225° C., preferably 180 to 220° C., preferably 185 to 215° C., preferably 190 to 210° C., more preferably 195 to 205° C., and yet more preferably about 200° C. In some embodiments, the refluxing occurs at a temperature of 190 to 210° C., preferably 191 to 209° C., preferably 192 to 208° C., preferably 193 to 207° C., preferably 194 to 206° C., preferably 195 to 205° C., preferably 196 to 204° C., preferably 197 to 203° C., preferably 198 to 202° C., more preferably 199 to 201° C., and yet more preferably about 200° C. In some embodiments, the reaction mixture during the refluxing is metal-free.

In some embodiments, the polyethylene terephthalate is from sources such as food containers, thermoform packaging, clamshell containers, polyester fibers, textile waste, industrial PET scrap, blister packs, cosmetic containers, PET trays, PET films, plastic sheets, pharmaceutical packaging, electronic device packaging, PET foils, strapping tapes, synthetic fabric waste, PET-coated papers, recycled garment fibers, nonwoven fabrics, bedding materials, PET-based insulation, curtain materials, carpet fibers, PET mesh, agricultural film waste, fleece fabric waste, pillow stuffing, mattress covers, shopping bags, protective sleeves, a combination thereof, and the like. In a preferred embodiment, the polyethylene terephthalate is from post-consumer water bottles. In an embodiment, PET flakes are obtained from shredding the post-consumer water bottles.

In other embodiments, a glycol including, but not limited to, diethylene glycol, triethylene glycol, propylene glycol, dipropylene glycol, tripropylene glycol, 1,3-propanediol, 1,4-butanediol, 2,3-butanediol, 1,5-pentanediol, neopentyl glycol, 1,6-hexanediol, 2-methyl-1,3-propanediol, 2-ethyl-1,3-hexanediol, 1,2-pentanediol, 2,4-pentanediol, cyclohexanedimethanol, polyethylene glycol, polypropylene glycol, trimethylene glycol, hexylene glycol, thiodiglycol, benzyl glycol, butyl glycol, glycerol, methylpropanediol, ethylbutyl glycol, isobutylene glycol, phenyl ethylene glycol, a combination thereof, and the like may be used in place of or in combination with the ethylene glycol.

The ionic liquid is selected from a group consisting of 1-butyl-3-methylimidazolium acetate ([BMIM][OAc]) and trihexyltetradecylphosphonium decanoate ([P6,6,6,14][Dec]). In other embodiments, an ionic liquid including, but not limited to, 1-ethyl-3-methylimidazolium chloride ([EMIM][Cl]), 1-butyl-3-methylimidazolium chloride ([BMIM][Cl]), 1-hexyl-3-methylimidazolium bromide ([HMIM][Br]), 1-octyl-3-methylimidazolium tetrafluoroborate ([OMIM][BF₄]), 1-ethyl-3-methylimidazolium tetrafluoroborate ([EMIM][BF₄]), 1-butyl-3-methylimidazolium hexafluorophosphate ([BMIM][PF₆]), 1-allyl-3-methylimidazolium chloride ([AMIM][Cl]), 1-ethyl-3-methylimidazolium acetate ([EMIM][OAc]), 1-benzyl-3-methylimidazolium chloride ([BnMIM][Cl]), choline chloride, choline acetate, choline lactate, tetramethylammonium acetate, tetrabutylammonium chloride, tetrabutylphosphonium bromide, trihexyl(tetradecyl)phosphonium chloride ([P6,6,6,14][Cl]), 1-butylpyridinium chloride ([BPy][Cl]), 1-methyl-3-octylimidazolium methylsulfate ([OMIM][MeSO₄]), 1-hexyl-3-methylimidazolium chloride ([HMIM][Cl]), 1-decyl-3-methylimidazolium bromide ([DMIM][Br]), 1-ethyl-3-methylimidazolium ethyl sulfate ([EMIM][EtSO₄]), 1-butyl-3-methylimidazolium hydrogen sulfate ([BMIM][HSO4]), 1-methylimidazolium nitrate ([MIM][NO3]), tributylmethylammonium methylsulfate, diethylmethylammonium triflate, triethylsulfonium methanesulfonate, 1-methyl-3-propylimidazolium dicyanamide ([MPIM][DCA]), 1-butyl-1-methylpyrrolidinium trifluoromethanesulfonate, N-methyl-N-propylpyrrolidinium dicyanamide trihexyltetradecylphosphonium bis(2,4,4-trimethylpentyl)phosphinate ([P6,6,6,14][TMPP]), a combination thereof, and the like may be used in combination with or in place of the ([BMIM][OAc]) and the ([P6,6,6,14][Dec]).

The ionic liquid is present in the reaction mixture in an amount of 2 to 15 percent by weight (wt. %), preferably 3 to 14 wt. %, preferably 4 to 13 wt. %, preferably 5 to 12 wt. %, preferably 6 to 11 wt. %, preferably 7 to 10 wt. %, and preferably 8 to 9 wt. % of the PET. In some embodiments, the ionic liquid is present in the reaction mixture in an amount of 4 to 6 wt. %, preferably 4.1 to 5.9 wt. %, preferably 4.2 to 5.8 wt. %, preferably 4.3 to 5.7 wt. %, preferably 4.4 to 5.6 wt. %, preferably 4.5 to 5.5 wt. %, preferably 4.6 to 5.4 wt. %, preferably 4.7 to 5.3 wt. %, preferably 4.8 to 5.2 wt. %, more preferably 4.9 to 5.1 wt. %, and yet more preferably about 5 wt. % of the PET. In a preferred embodiment, the ionic liquid is present in the reaction mixture in an amount of 5 wt. % of the PET.

In some embodiments, the refluxing occurs for 1 to 5 hours, preferably 1.5 to 4.5 hours, preferably 2 to 4 hours, more preferably 2.5 to 3.5 hours, and yet more preferably about 3 hours. In a preferred embodiment, the refluxing occurs for 3 hours. In some embodiments, the refluxing occurs at 0.8 to 1.2 bar, preferably 0.85 to 1.15 bar, preferably 0.9 to 1.1 bar, more preferably 0.95 to 1.05 bar, and yet more preferably about 1.0 bar. In a preferred embodiment, the refluxing occurs at atmospheric pressure.

In some embodiments, the weight ratio of the PET to the ethylene glycol during the refluxing is 1-3:20-25, preferably 1.2-2.8:20.5-24.5, preferably 1.5-2.5:21-24, more preferably 1.8-2.2:21.5-23.5, and yet more preferably about 2:22. In a preferred embodiment, a weight ratio of the PET to the ethylene glycol is 1:10.

At step 54, the method 50 includes filtering the reaction mixture to collect a filtrate. In some embodiments, the filtration technique used to filter the reaction mixture includes, but is not limited to, gravity filtration, hot filtration, cold filtration, pressure filtration, vacuum filtration, membrane filtration, syringe filtration, centrifugal filtration, depth filtration, cross-flow filtration, microfiltration, ultrafiltration, nanofiltration, reverse osmosis, sintered glass filtration, paper filtration, fritted disc filtration, cotton plug filtration, sand filtration, gel filtration, dialysis filtration, magnetic filtration, oil filtration, carbon filtration, ceramic filtration, resin filtration, screen filtration, hemodialysis filtration, biosand filtration, percolation filtration, electrofiltration, a combination thereof, and the like. In a preferred embodiment, the filtration technique is vacuum filtration.

At step 56, the method 50 includes cooling the filtrate to form bis(2-hydroxyethyl) terephthalate (BHET). In some embodiments, the cooling occurs at a temperature of 2 to 6° C., preferably 2.5 to 5.5° C., preferably 3 to 5° C., more preferably 3.5 to 4.5° C., and more preferably about 4° C. In a preferred embodiment, the cooling temperature is 4° C. In some embodiments, the cooling occurs for 16 to 24 hours, preferably 17 to 23 hours, preferably 18 to 22 hours, more preferably 19 to 21 hours, and yet more preferably about 20 hours. In a preferred embodiment, cooling occurs for 20 hours.

At least 95 percent by weight (wt. %), preferably at least 95.5 wt. %, preferably at least 96 wt. %, preferably at least 96.5 wt. %, preferably at least 97 wt. %, preferably at least 97.5 wt. %, preferably at least 98 wt. %, preferably at least 98.5 wt. %, preferably at least 99 wt. %, and preferably at least 99.5 wt. % of the PET is converted to BHET. In a preferred embodiment, at least 97 wt. % of the PET is converted to BHET. In another preferred embodiment, at least 99 wt. % of the PET is converted to BHET. In yet another preferred embodiment, about 100 wt. % of the PET is converted to BHET.

In some embodiments, the ionic liquid is [BMIM][OAc] and the BHET has a yield percentage of 30 to 35%, preferably 30.5 to 34.5%, preferably 31 to 34%, preferably 31.5 to 33.5%, and preferably 32 to 33% based on an initial weight of the PET. In some embodiments, the ionic liquid is [BMIM][OAc] and the BHET has a yield percentage of 33 to 34%, preferably 33.1 to 33.9%, preferably 33.2 to 33.8%, preferably 33.3 to 33.7%, preferably 33.4 to 33.65%, and more preferably 33.5 to 33.6% based on an initial weight of the PET. In a preferred embodiment, the ionic liquid is [BMIM][OAc] and the BHET has a yield percentage of about 33.58% based on an initial weight of the PET.

In some embodiments, the ionic liquid is [P6,6,6,14][Dec] and the BHET has a yield percentage of 70 to 75%, preferably 70.5 to 74.5%, preferably 71 to 74%, preferably 71.5 to 73.5%, and preferably 72 to 73% based on an initial weight of the PET. In some embodiments, the ionic liquid is [P6,6,6,14][Dec] and the BHET has a yield percentage of 72 to 73%, preferably 72.1 to 72.9%, preferably 72.2 to 72.8%, preferably 72.3 to 72.7%, and preferably 72.4 to 72.6% based on an initial weight of the PET. In a preferred embodiment, the ionic liquid is [P6,6,6,14][Dec] and the BHET has a yield percentage of about 72.4% based on an initial weight of the PET.

In some embodiments, the ionic liquid is trihexyltetradecylphosphonium bis(2,4,4-trimethylpentyl)phosphinate ([P6,6,6,14][TMPP]) and 55 to 65%, preferably 56 to 64%, preferably 57 to 63%, preferably 58 to 62%, more preferably 59 to 61%, more preferably about 60% of the PET is converted to the BHET based on an initial weight of the PET. In some embodiments, the ionic liquid is trihexyltetradecylphosphonium bis(2,4,4-trimethylpentyl)phosphinate ([P6,6,6,14][TMPP]) and 59 to 61%, preferably 59.1 to 60.9%, preferably 59.2 to 60.8%, preferably 59.3 to 60.7%, preferably 59.4 to 60.6%, preferably 59.5 to 60.5%, preferably 59.6 to 60.4%, preferably 59.7 to 60.3%, preferably 59.8 to 60.2%, more preferably 59.9 to 60.1%, and yet more preferably about 60% of the PET is converted to the BHET based on an initial weight of the PET. In a preferred embodiment, the ionic liquid is trihexyltetradecylphosphonium bis(2,4,4-trimethylpentyl)phosphinate ([P6,6,6,14][TMPP]) and 60% of the PET is converted to the BHET based on an initial weight of the PET.

In some embodiments, the method further includes mixing the BHET to a temperature of 150 to 250° C. under nitrogen to melt the BHET. At step 58, the method 50 includes mixing the BHET to a temperature of 150 to 250° C., preferably 160 to 240° C., preferably 170 to 230° C., preferably 180 to 220° C., more preferably 190 to 210° C., and yet more preferably about 200° C. In a preferred embodiment, the temperature to melt the BHET is 200° C.

At step 60, the method 50 includes heating the melted BHET to a temperature of 250 to 300° C., preferably 255 to 295° C., preferably 260 to 290° C., more preferably 265 to 285° C., and yet more preferably 270 to 280° C. under vacuum for 40 to 60 minutes, preferably 42 to 58 minutes, preferably 44 to 56 minutes, preferably 46 to 54 minutes, more preferably 48 to 52 minutes, and yet more preferably about 50 minutes to form a polycondensation product. In a preferred embodiment, heating the melted BHET to a temperature of 270° C. under vacuum for 50 minutes forms a polycondensation product. In some embodiments, the polycondensation product is the polyethylene terephthalate (PET). In an embodiment, the BHET is repolymerized into the PET. In some embodiments, the BHET self-condenses into the PET.

The following examples are provided solely for illustration and are not to be construed as limitations of the present disclosure, as many variations thereof are possible without departing from the spirit and scope of the present disclosure.

### Examples

The following examples describe and demonstrate a method of polyethylene terephthalate glycolysis. The examples are provided solely for illustration and are not to be construed as limitations of the present disclosure, as many variations thereof are possible without departing from the spirit and scope of the present disclosure.

### Example 1: Materials

Anhydrous ethylene glycol (EG) having a purity of 99.8% 1-allyl-3-methylimidazolium chloride ([AMIM]CI), 1-butyl-3-methylimidazolium acetate ([BMIM][OAc]), trihexyltetradecylphosphonium decanoate ([P6,6,6,14][Dec]), trihexyltetradecylphosphonium chloride ([P6,6,6,14]Cl), trihexyltetradecylphosphonium bis(2,4,4-trimethylpentyl)phosphinate ([P6,6,6,14][TMPP]), and methyltrioctylammonium chloride ([MTOA]Cl) were obtained from Sigma Aldrich and used without further purification. Polyethylene terephthalate (PET) flakes were prepared by mechanical shredding of post-consumer water bottles.

### Example 2: Glycolysis of Polyethylene Terephthalate (PET)

Glycolysis reactions were conducted in a two-step process involving reflux heating followed by recrystallization. In the reflux stage, 2.0 grams of PET flakes and 20.0 milliliters of ethylene glycol were introduced into a 250-milliliter round-bottom (RB) flask reactor which was heated to and maintained at 200° C. using an oil bath. An ionic liquid, in the range of 5 to 10% by weight relative to the PET mass, was added to the reactor prior to thermal equilibration. The reactions were subject to continuous reflux for a duration of 3 hours under atmospheric pressure. A schematic representation of the glycolysis setup and procedure is provided in FIG. 5. Unreacted PET was collected by rapid filtration, rinsed with water, dried, and measured to calculate the conversion by equation 1:

$$Conversion ( \% ) = \frac{{m}_{PET} ( initial ) - {m}_{PET} ( final )}{{m}_{PET} ( initial )} \times 100 \qquad ( 1 )$$

In the recrystallization step, the filtrate was stored in the fridge at 4.0° C. for 20 hours. White crystals of BHET appeared overnight and were filtered off. The obtained white product was dried and measured to calculate the yield of the product:

$$Yield ( \% ) = \frac{{m}_{BHET} ( obtained )}{{m}_{BHET} ( expected )} \times 100 \qquad ( 2 )$$

$$where :$$

$${m}_{BHET} ( expected ) = \frac{{m}_{PET} ( initial )}{{M}_{PET}} \times {M}_{BHET} \qquad ( 3 )$$

$${M}_{PET} = 19 2.17 \frac{g}{mol}$$

$${M}_{BHET} = 25 4.24 \frac{g}{mol}$$

### Example 3: Re-Polymerization of Bis(2-Hydroxyethyl) Terephthalate (BHET)

3.0 grams of bis(2-hydroxyethyl) terephthalate (BHET), obtained from the glycolysis reactions described above, was transferred into a conical flask equipped with a magnetic stirring unit. The flask was connected to a distillation assembly and subjected to heating under a continuous flow of nitrogen gas. The temperature was initially maintained at 200° C. until complete melting of the BHET occurred. Upon melting, the temperature was elevated to 270° C., the nitrogen flow was discontinued, and a vacuum was applied to the system. Post maintaining vacuum condition for 50 minutes at 270° C., heating was terminated, and the reaction mixture was quenched with deionized water. The resulting solid was collected by filtration and subsequently dried under vacuum at 100° C. The solid polycondensation product was characterized by thermogravimetric analysis (TGA), Fourier-transform infrared (FTIR) spectroscopy, and nuclear magnetic resonance (NMR) spectroscopy. The physicochemical and spectral characteristics of the obtained polymer were consistent with those of the original polyethylene terephthalate (PET) flakes, confirming successful re-polymerization.

Various ionic liquids including 1-butyl-3-methylimidazolium acetate ([BMIM][OAc]) and trihexyltetradecylphosphonium decanoate ([P6,6,6,14][Dec]) were identified as effective catalysts in the glycolysis of polyethylene terephthalate (PET), each resulting in complete conversion of PET to bis(2-hydroxyethyl) terephthalate (BHET). The ionic liquid [P6,6,6,14][TMPP] achieved a PET conversion rate of approximately 60%; however, the final product could not be isolated due to the presence of high levels of impurities in the reaction mixture. Other evaluated ionic liquids, including [AMIM]Cl, [P6,6,6,14]Cl, and [MTOA]Cl, were found to be catalytically inactive or ineffective under the same reaction conditions.

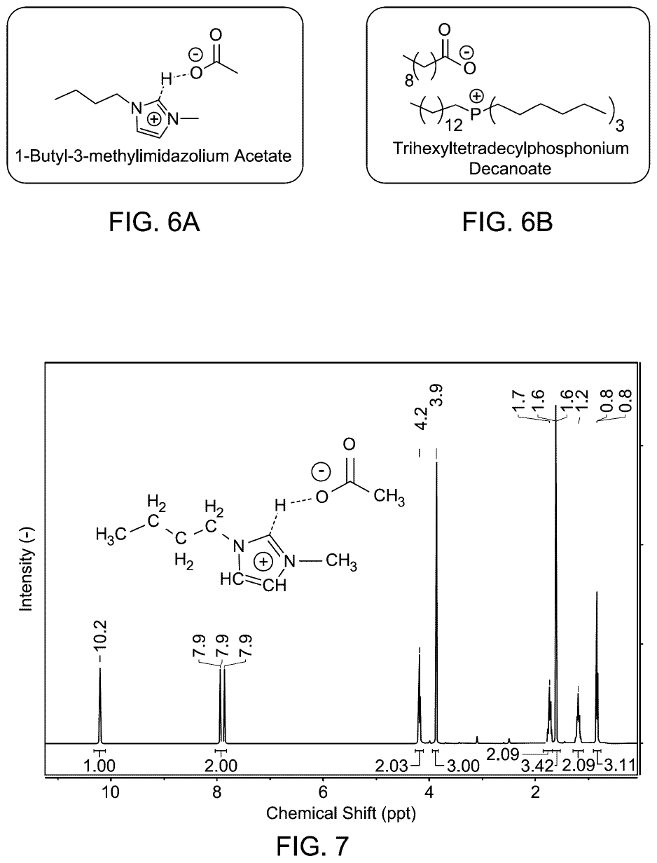

FIG. 6A and FIG. 6B depict molecular structures of [BMIM][OAc] and [P6,6,6,14][TMPP], respectively. Characterization of [BMIM][OAc] was carried out using proton nuclear magnetic resonance (¹H NMR) spectroscopy, as illustrated in FIG. 7. A singlet resonance at 10.2 ppm was assigned to the acidic proton located at the C2 position of the imidazolium ring, which is de-shielded due to electron-withdrawing influence of adjacent nitrogen atoms. The doublet peak at 7.9 ppm was attributed to the aromatic protons at C4 and C5 positions of the imidazolium ring, both appearing downfield under the influence of the ring's positive charge of the nitrogen. The triplet peak at 4.2 ppm corresponds to the methylene (—CH₂—) group attached to the butyl chain bonded directly to the nitrogen atom of the imidazolium ring, and this peak is slightly de-shielded due to its proximity to the positively charged nitrogen. The sharp singlet at 3.9 ppm represents the methyl group (—CH₃) attached to the nitrogen on the imidazolium ring, where the positive charge on the nitrogen causes slight de-shielding of the methyl group. The peaks located at around 1.7 ppm corresponds to the methylene group (—CH₂—) that is adjacent to the methylene group directly attached to the imidazolium ring, and these protons are more shielded as they move further from the influence of the charged ring. The peaks at 1.1 ppm and 0.8 ppm correspond to the terminal methylene (—CH₂—) and the methyl group (—CH₃) on the butyl chain, respectively, with the terminal methyl group being the most shielded, which results in an up-field chemical shift.

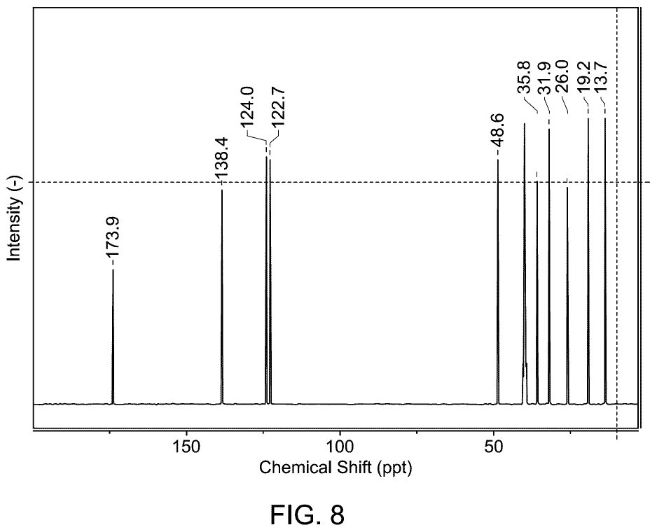

The carbon environments of [BMIM][OAc] were confirmed by ¹³C NMR spectroscopy, as shown in FIG. 8. The peak at 173.9 ppm was attributed to the carbonyl carbon of the acetate anion, which is highly de-shielded due to its attachment to electronegative oxygen atoms. The peak at 138.4 ppm was assigned to the C2 carbon which is bonded to two nitrogen atoms and is more de-shielded. The peaks in the 122.7 ppm to 124.0 ppm range corresponded to C4 and C5 carbons of the imidazolium ring. These carbons are less de-shielded compared to C2 but still experience the influence of the adjacent nitrogen atoms. The peak at 48.6 ppm was associated with the methylene carbon attached directly to the imidazolium nitrogen, as this carbon is relatively close to the positive charge of the imidazolium ring. Additional resonances at 31.9 ppm, 26.0 ppm, 19.2 ppm, and 13.7 ppm were attributed to the butyl chain carbons. The ¹³C NMR spectrum was consistent with the carbon framework of [BMIM][OAc], with each carbon resonance showing up according to the level of shielding or de-shielding effects experienced by the respective carbons.

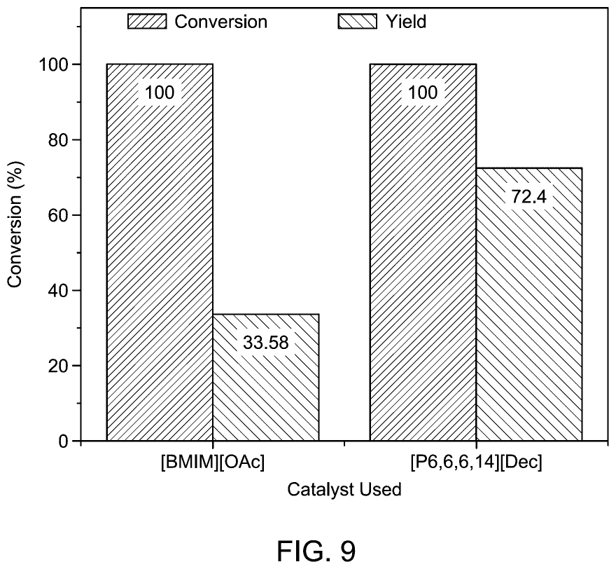

Results of polyethylene terephthalate (PET) glycolysis using various ionic liquids under standardized conditions are shown in FIG. 9. The reactions were conducted at 200° C. using 5 wt. % ionic liquid relative to PET mass, with a reaction duration of 3 hours. Under these conditions, 1-butyl-3-methylimidazolium acetate ([BMIM][OAc]) and trihexyltetradecylphosphonium decanoate ([P6,6,6,14][Dec]) were identified as the most catalytically active systems, each achieving complete (100%) PET conversion. The use of [BMIM][OAc] resulted in a bis(2-hydroxyethyl) terephthalate (BHET) yield of 33.58 wt. %, whereas [P6,6,6,14][Dec] achieved a BHET yield of 72.4 wt. %, indicating a higher selectivity toward the target monomer under identical conditions. The ionic liquid trihexyltetradecylphosphonium bis(2,4,4-trimethylpentyl)phosphinate ([P6,6,6,14][TMPP]) enabled approximately 60% PET conversion; however, isolation of the final product was unsuccessful due to the presence of impurities in the reaction matrix. The ionic liquids 1-allyl-3-methylimidazolium chloride ([AMIM]Cl), trihexyltetradecylphosphonium chloride ([P6,6,6,14]Cl), and methyltrioctylammonium chloride ([MTOA]Cl) exhibited no observable catalytic activity for PET depolymerization under the tested conditions.

Qualitative identification and confirmation of the glycolysis product were carried out using Fourier-transform infrared (FTIR) spectroscopy, nuclear magnetic resonance (NMR) spectroscopy, and gas chromatography-mass spectrometry (GC-MS). The collected spectral and chromatographic data confirmed the formation of bis(2-hydroxyethyl) terephthalate (BHET) as the principal product.

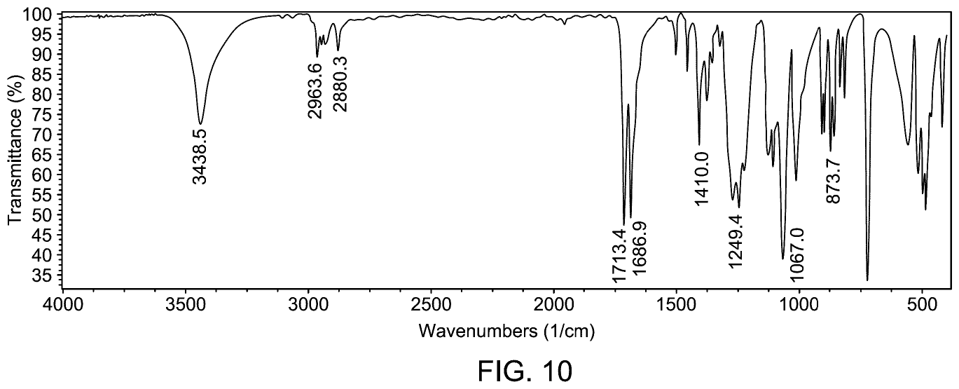

FTIR spectroscopy was used to confirm the chemical identity of the bis(2-hydroxyethyl) terephthalate (BHET) product obtained from polyethylene terephthalate (PET) glycolysis. The FTIR spectrum is shown in FIG. 10. A strong O—H stretching vibration observed near 3400 cm⁻¹ indicated the presence of hydroxyl functional groups. Absorption bands in the range of 2880 cm⁻¹ to 3100 cm⁻¹ corresponded to alkyl C—H stretching vibrations, which support the presence of aromatic ring structures [Tang, Y. et al., A low-temperature ionic liquid demulsifier derived from recycled PET waste plastics, J. Mol. Liq., 2024, 394, 123737, which is incorporated herein by reference in its entirety]. A pronounced C═O stretching band detected around 1710 cm⁻¹ is characteristic of ester carbonyl functionalities, while the C—O stretching vibration at approximately 1060 cm⁻¹ is indicative of ester linkages found in BHET [Yu, Y. et al., Ti—Si composite glycol salts: depolymerization and repolymerization studies of PET, RSC Adv., 2023, 13, 36337-36345, which is incorporated herein by reference in its entirety]. Absorptions in the region near 870 cm⁻¹ are attributed to aromatic ring vibrations. The combined spectral features are consistent with the formation of BHET [Liu, Y. et al., Degradation of poly(ethylene terephthalate) catalyzed by metal-free choline-based ionic liquids, Green Chem., 2020, 22, 3122-3131, which is incorporated herein by reference in its entirety].

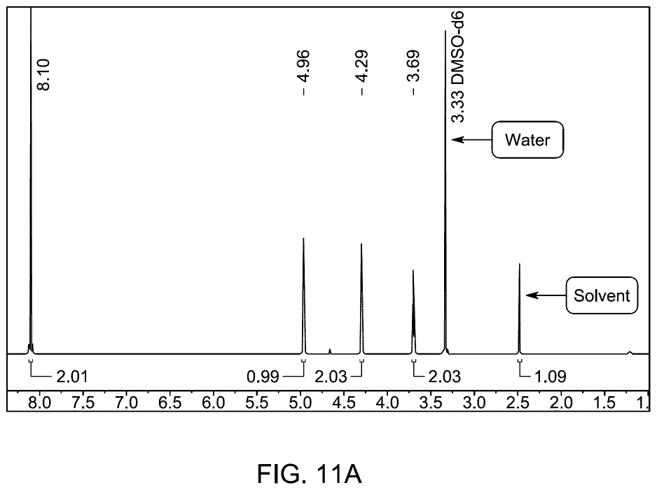

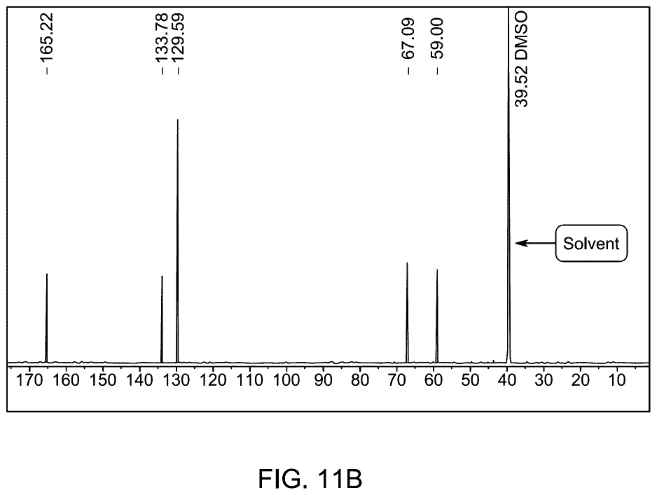

Further structural confirmation was conducted using proton and carbon nuclear magnetic resonance (¹H NMR and ¹³C NMR) spectroscopy, as shown in FIG. 11A and FIG. 11B, respectively. In the ¹H NMR spectrum, a singlet at δ=8.1 ppm was assigned to the aromatic protons, which appear downfield due to the electron-withdrawing ester groups and resonance effects of the aromatic system [Imran, M. et al., Manganese-, cobalt-, and zinc-based mixed-oxide spinels as novel catalysts for the chemical recycling of poly(ethylene terephthalate) via glycolysis, Polym. Degrad. Stab., 2013, 98, 904-915, which is incorporated herein by reference in its entirety]. A triplet at δ=4.96 ppm was attributed to the hydroxyl proton, de-shielded due to the electronegativity of the adjacent oxygen atom. A second triplet at δ=4.29 ppm was assigned to the methylene group adjacent to the ester carbonyl, experiencing de-shielding from the nearby carbonyl moiety. A quartet at δ=3.69 ppm was attributed to the methylene group adjacent to the hydroxyl group, exhibiting an up-field shift. The integration ratios of the observed signals confirm the formation of a high-purity BHET product [Bhanderi, K. K. et al., Optimization process for glycolysis of poly(ethylene terephthalate) using bio-degradable & recyclable heterogeneous catalyst, J. Indian Chem. Soc., 2023, 100, 100904, which is incorporated herein by reference in its entirety].

The ¹³C NMR spectrum of bis(2-hydroxyethyl) terephthalate (BHET), shown in FIG. 11B, confirms the carbon environments of the glycolysis product. A peak at δ=165.22 ppm was assigned to the ester carbonyl carbon (—COO—), which is de-shielded due to the electron-withdrawing influence of the adjacent oxygen atoms. Peaks at δ=133.78 ppm and δ=129.59 ppm correspond to the aromatic ring carbons (—C₆H₄—), which exhibit downfield shifts due to conjugation with the ester functional groups. Signals at δ=67.09 ppm and δ=59.00 ppm were assigned to two distinct methylene (—CH₂—) groups. The chemical shift difference arises from the varying proximity of the methylene groups to electron-withdrawing ester and hydroxyl functionalities. The observed ¹³C NMR spectrum agrees with the BHET structure.

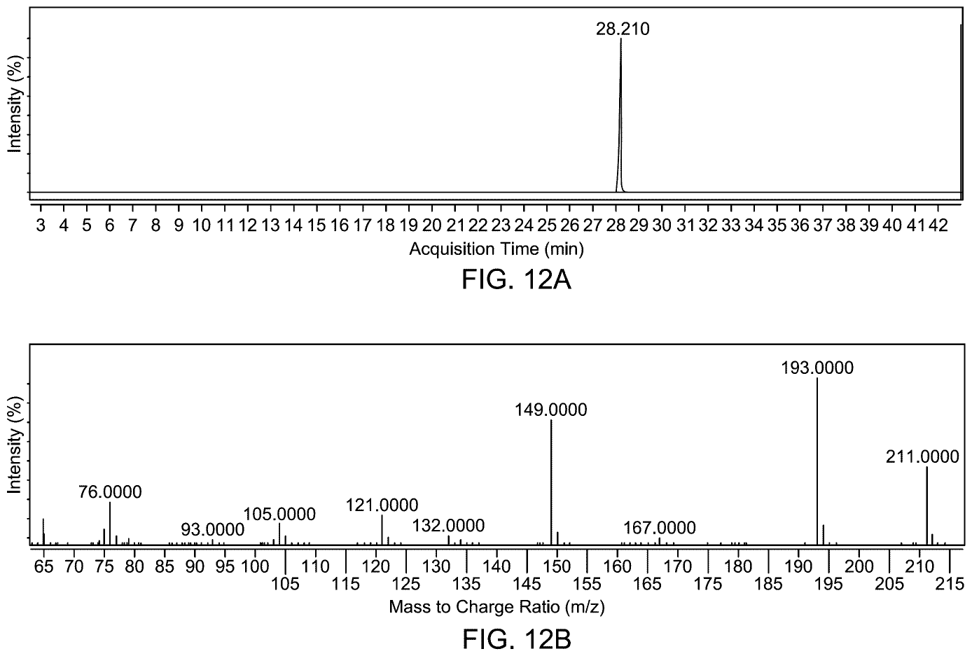

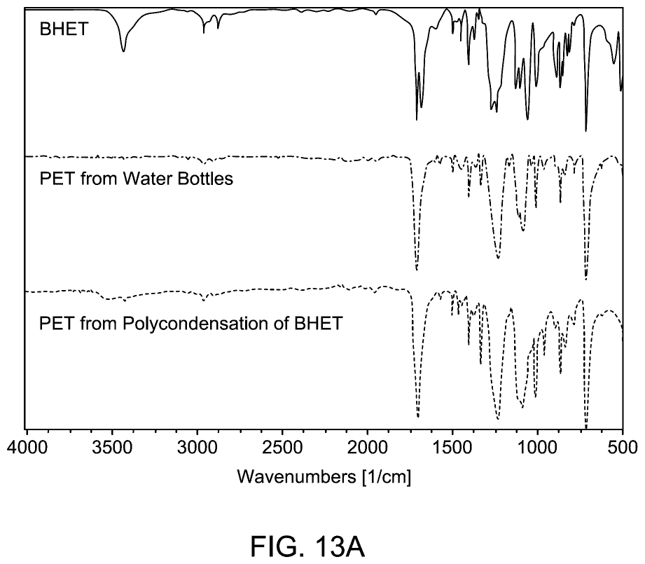

GC-MS analysis was conducted to further validate the structure, purity, and molecular identity of BHET. The GC-MS chromatogram and mass spectrum are presented in FIGS. 12A-12B, respectively, with the gas chromatography trace shown in FIG. 12A. The chromatographic profile indicated a single major peak, confirming the high purity of the isolated BHET product. The mass spectrum of the corresponding chromatographic peak revealed a molecular ion at m/z 211, corresponding to C₁₀H₉O₅, which represents the BHET molecule with one terminal ethylene group eliminated [Bhanderi, K. K. et al., Optimization process for glycolysis of poly(ethylene terephthalate) using bio-degradable & recyclable heterogeneous catalyst, J. Indian Chem. Soc., 2023, 100, 100904, which is incorporated herein by reference in its entirety]. A signal at m/z 193 was attributed to the loss of a complete ethylene glycol unit. A prominent fragment at m/z 149 was assigned to a terephthalate core unit with one hydroxyl group removed (C₈H₃O₃). Additional peaks at 121 m/z, 104 m/z, and 76 m/z were assigned to C₇H₄O₂·, C₇H₄O₂·, and C₆H₄^{2·} fragments, respectively. The fragmentation pattern corresponds to the breakdown of PET and supports the successful synthesis of BHET.

The BHET monomers obtained from individual glycolysis reactions were combined and subjected to polycondensation without additional purification steps. The resulting polymer was analyzed and compared to the original PET and the BHET monomer using FTIR spectroscopy, thermogravimetric analysis (TGA), and solid-state nuclear magnetic resonance (NMR) spectroscopy.

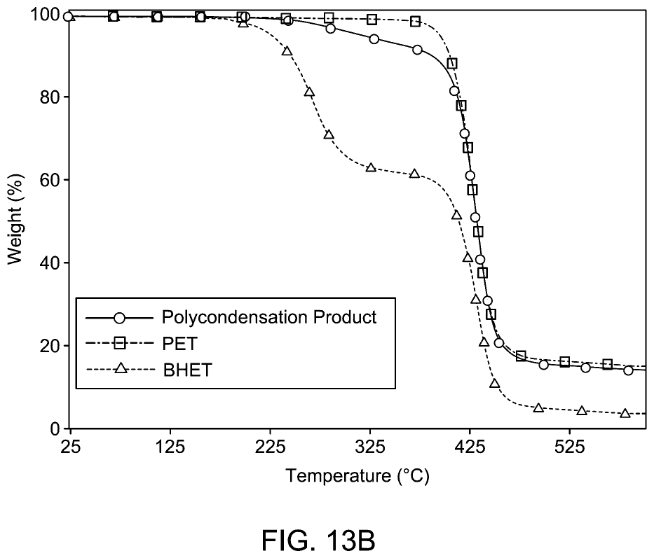

FIG. 13A displays the FTIR spectra for BHET, PET, and the polycondensation product. The broad absorption band near 3500 cm⁻¹ observed in the BHET spectrum, attributed to hydroxyl (—OH) stretching vibrations, was absent in the spectrum of the polycondensation product. Additionally, a high degree of spectral similarity was observed between the polycondensation product and PET derived from commercial water bottles, indicating successful re-polymerization of BHET into polyethylene terephthalate. Furthermore, FIG. 13B presents a comparative thermogravimetric analysis of BHET, PET, and the re-polymerized product. The thermal degradation profile of the polycondensation product closely resembled that of the PET reference and was different from that of BHET. These findings confirm that the re-polymerized material exhibits thermal stability and decomposition characteristics similar to the pure PET.

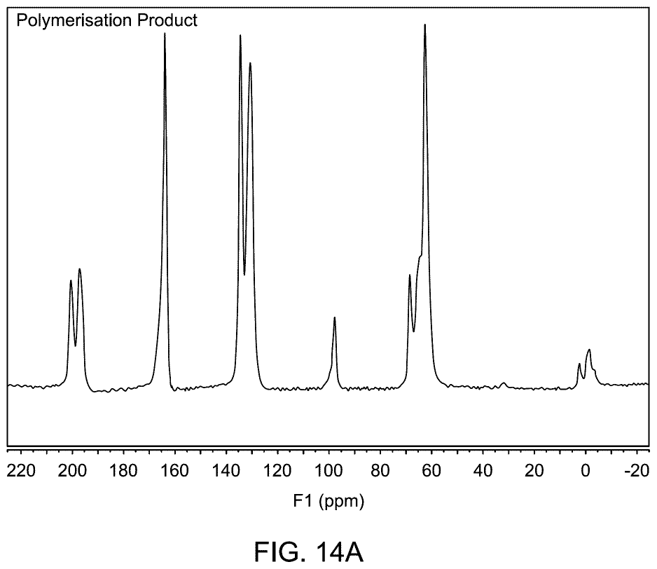

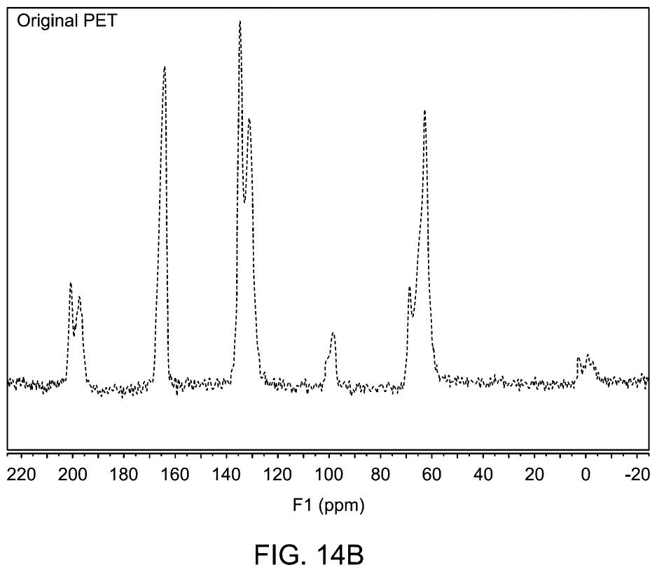

Solid-state 13C NMR spectroscopy was employed to confirm the structural identity of the re-polymerized product obtained from bis(2-hydroxyethyl) terephthalate (BHET). FIG. 14 presents a comparative analysis of the 13C NMR spectra for the polycondensation product and the original polyethylene terephthalate (PET) material. The spectrum of the polymerization product exhibited chemical shift patterns and resonance intensities closely matching those of the original PET, thereby indicating successful regeneration of the PET chemical structure through the polycondensation process.

The present disclosure provides a method for the glycolysis of polyethylene terephthalate (PET) using metal-free ionic liquid catalysts, including [BMIM][OAc] and [P6,6,6,14][Dec], resulting in 100% PET conversion. The ionic liquid [P6,6,6,14][Dec] achieved a BHET yield of up to 72.4% and [BMIM][OAc] yielded 33.58%, both demonstrating high catalytic activity and selectivity toward bis(2-hydroxyethyl) terephthalate (BHET) formation. The BHET obtained from glycolysis was successfully repolymerized to generate recycled PET (rPET), as confirmed by FTIR, TGA, and NMR analyses. The resulting rPET exhibited physicochemical properties comparable to those of pure PET. The disclosed method offers a metal-free and sustainable approach to PET depolymerization and repolymerization, contributing to circular plastic economy efforts and addressing the environmental burden of plastic waste.

Numerous modifications and variations of the present disclosure are possible in light of the above teachings. It is therefore to be understood that within the scope of the appended claims, the disclosure may be practiced otherwise than as specifically described herein.

1: A method of polyethylene terephthalate glycolysis, comprising:
refluxing a glycolysis mixture comprising a polyethylene terephthalate (PET), ethylene glycol, and an ionic liquid at a temperature of 100 to 300° C. to form a reaction mixture,
wherein the polyethylene terephthalate is from post-consumer water bottles,
wherein the ionic liquid is selected from a group consisting of 1-butyl-3-methylimidazolium acetate ([BMIM][OAc]) and trihexyltetradecylphosphonium decanoate ([P6,6,6,14][Dec]),
wherein the ionic liquid is present in the reaction mixture in an amount of 2 to 15 percent by weight (wt. %) of the PET,
filtering the reaction mixture to collect a filtrate; and
cooling the filtrate to form bis(2-hydroxyethyl) terephthalate (BHET),
wherein at least 95 percent by weight (wt. %) of the PET is converted to BHET.

2: The method of claim of 1, wherein the ionic liquid is [BMIM][OAc] and the BHET has a yield percentage of 30 to 35% based on an initial weight of the PET.

3: The method of claim 1, wherein the ionic liquid is [P6,6,6,14][Dec] and the BHET has a yield percentage of 70 to 75% based on an initial weight of the PET.

4: The method of claim 1, wherein the refluxing occurs for 1 to 5 hours.

5: The method of claim 1, wherein the cooling occurs at a temperature of 2 to 6° C.

6: The method of claim 1, wherein the cooling occurs for 16 to 24 hours.

7: The method of claim 1, wherein the ionic liquid is present in the reaction mixture in an amount of 4 to 6 wt. % of the PET.

8: The method of claim 1, further comprising:
mixing the BHET to a temperature of 150 to 250° C. under nitrogen to melt the BHET; and
heating the melted BHET to a temperature of 250 to 300° C. under vacuum for 40 to 60 minutes to form a polycondensation product,
wherein the polycondensation product is the polyethylene terephthalate (PET).

9: The method of claim 1, wherein the ionic liquid is trihexyltetradecylphosphonium bis(2,4,4-trimethylpentyl)phosphinate ([P6,6,6,14][TMPP]) and wherein 55 to 65% of the PET is converted to the BHET based on an initial weight of the PET.

10: The method of claim 1, wherein the refluxing occurs at 0.8 to 1.2 bar.

11: The method of claim 1, wherein a weight ratio of the PET to the ethylene glycol during the refluxing is 1-3:20-25.

12: The method of claim 1, wherein at least 97 wt. % of the PET is converted to BHET.

13: The method of claim 1, wherein at least 99 wt. % of the PET is converted to BHET.

14: The method of claim of 1, wherein the ionic liquid is [BMIM][OAc] and the BHET has a yield percentage of 33 to 34% based on an initial weight of the PET.

15: The method of claim 1, wherein the ionic liquid is [P6,6,6,14][Dec] and the BHET has a yield percentage of 72 to 73% based on an initial weight of the PET.

16: The method of claim 1, wherein the reaction mixture during the refluxing is metal-free.

17: The method of claim 1, wherein the ionic liquid is trihexyltetradecylphosphonium bis(2,4,4-trimethylpentyl)phosphinate ([P6,6,6,14][TMPP]) and wherein 59 to 61% of the PET is converted to the BHET based on an initial weight of the PET.

18: The method of claim 1, wherein the refluxing occurs at a temperature of 150 to 250° C.

19: The method of claim 8, further comprising:
polymerizing the BHET to form PET.

20: The method of claim 1, wherein the refluxing occurs at a temperature of 190 to 210° C.
